SEQUENCE LISTING (4 sequences)

SEQ ID NO: 1 - protein, 242 aa, Homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 2 - protein, 122 aa, Homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 3 - protein, 512 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 4 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

(19) United States
(12) Patent Application Publication
(10) Pub. No.: US 2022/0137054 A1
(43) Pub. Date: May 5, 2022

(54) NEW BIOMARKERS AND BIOTARGETS IN RENAL CELL CARCINOMA

(71) Applicants: INSERM (Institut National de la Santé et de la Recherche Médicale), Paris (FR); Université de Bordeaux, Bordeaux (FR); Centre National de la Recherche Scientifique (CNRS), Paris (FR); Université Cote d'Azur, Nice (FR); University of Liverpool, Liverpool (GB)

(72) Inventors: Andreas BIKFALVI, Pessac (FR); Lindsay COOLEY, Pessac (FR); Wilfried SOULEYREAU, Pessac (FR); Gilles PAGES, Nice (FR); Francesco FALCIANI, Liverpool (GB); Maeva DUFIES, Monaco (MC); Kim CLARKE, Liverpool (GB)

(21) Appl. No.: 17/434,225

(22) PCT Filed: Mar. 4, 2020
(86) PCT No.: PCT/EP2020/055649

§ 371 Date: Aug. 26, 2021

(30) Foreign Application Priority Data
Mar. 5, 2019 (EP) 19305252.9

(51) Int. Cl. G01N 33/574; C12Q 1/6886; G01N 33/68

(52) U.S. Cl. CPC G01N 33/57438; C12Q 1/6886; G01N 33/6869; C12Q 2600/158; C12Q 2600/118; C12Q 2600/106; C12N 2320/30

(57) ABSTRACT

Renal Cell Carcinoma (RCC) encompasses a heterogeneous group of cancers derived from renal tubular epithelial cells and has a worldwide mortality. However, mortality rates have barely improved over the last 20 years. Novel biomarkers and biomarkers are thus urgently required for this cancer. The inventors have devised a strategy to produce mouse cancer cell lines of progressively enhanced aggressiveness and specialization. The mouse renal cancer cell line RENCA was serially passaged in vivo using multiple implantation strategies designed to replicate different aspects of primary tumour growth and metastasis. Transcriptomic and epigenomic data has been acquired for the derived cell lines and primary analyses have been performed. The inventors then selected plurality of genes with no reported role in RCC which were upregulated in their specialized cell lines, and checked their relevance in patient data and clinical samples. This approach contributes to identify 4 serum biomarkers, namely IL-34, SAA2, PONL1 and CFB that are suitable for predicting survival time in patients suffering from RCC. The inventors also validated that the 4 proteins are also biotargets for the treatment of RCC.

### FIELD OF THE INVENTION

The present relates to new biomarkers and biotargets in renal cell carcinoma.

### BACKGROUND OF THE INVENTION

Renal Cell Carcinoma (RCC) encompasses a heterogeneous group of cancers derived from renal tubular epithelial cells and has a worldwide mortality of over 140,000 people per year. The disease encompasses multiple histological and molecular subtypes, of which clear cell RCC (ccRCC) is the most common. The incidence and prevalence of RCC are rising, along with increases in related risk factors such as hypertension, diabetes and obesity¹. However, mortality rates have barely improved over the last 20 years according to Surveillance, Epidemiology and End Results (SEER) data. Gandaglia et al² report a continuing upward trend in both incidence and mortality even in patients with localized disease. These data are in stark contrast with markedly improving survival rates in many other cancers and highlights RCC as one of the cancers in which current therapeutic approaches have failed to make the advances hoped for. Novel approaches to this problem are thus urgently required. When disease is localized to the kidney, surgical resection is the preferred option. However, therapeutic options for metastatic disease are limited. ccRCC metastasizes primarily to the lungs (secondarily to liver and bone), and 5 year survival is less than 10%^{3,4,5}. Furthermore, 40% of patients with seemingly localized disease will also relapse later with localized or metastatic disease. Localised recurrence is also difficult to treat, difficult to predict, and has a poor prognosis^{6,7}.

The challenges associated with treatment of RCC include high levels of resistance to traditional chemotherapeutic drugs⁸. The majority of currently available targeted therapies focus on inhibiting angiogenesis driven by the VEGF/VEGFR axis⁹. While progress has been made at extending life somewhat, such therapies are rarely curative, and will act primarily to “inhibit” the disease making eventual drug resistance almost inevitable. The high cost and failure rate of this approach is significant. Second line treatments include mTOR inhibitors and immunotherapeutic agents¹⁰. These can be successful, but are effective for only a limited subset of patients¹¹. The pathophysiology of RCC still far from understood, and there is a clear need to identify key mechanisms in RCC progression in order to open up novel therapeutic avenues targeting different aspects of RCC biology. Furthermore, clinical treatment of RCC is hampered by a lack of relevant biomarkers. Currently, no fully validated molecular biomarkers for RCC are in clinical practice. Response to currently available treatments and long term disease free survival is highly variable and problematic to predict. Patient diagnosis, prognosis, and clinical decisions are currently made based on histological information such as Fuhrman grade and tumour stage. Therapy selection is based on limited guidelines and response to previous treatments. In this respect, clinical treatment of RCC lags behind other cancers for which molecular knowledge is invaluable in guiding clinical decisions e.g. hormone receptor status in breast cancer.

### SUMMARY OF THE INVENTION

The present relates to new biomarkers and biotargets in renal cell carcinoma. In particular, the present invention is defined by the claims.

### DETAILED DESCRIPTION OF THE INVENTION

Methods for Predicting the Survival Time and Uses Thereof:

The first object of the present invention relates to a method for predicting the survival time of a patient suffering from a renal cell carcinoma (RCC) comprising i) determining the expression level of at least one biomarker selected from the group consisting of IL-34, SAA2, PONL1 and CFB in a sample obtained from the patient, ii) comparing the expression level determined at step i) with a predetermined reference value and wherein a difference between the determined expression level and said predetermined reference value is indicative whether the patient will have a long or short survival time.

As used herein, the term “renal cell carcinoma” or “RCC” has its general meaning in the art and refers to refers to a cancer originated from the renal tubular epithelial cells in the kidney. According to the pathological features, the cancer is classified into clear cell type, granular cell type, chromophobe type, spindle type, cyst-associated type, cyst-originating type, cystic type, or papillary type. In some embodiments, the renal cell carcinoma (RCC) is at Stage I, II, III, or IV as determined by the TNM classification, but however the present invention is accurately useful for predicting the survival time of patients when said cancer has been classified as Stage II or III by the TNM classification, i.e. non metastatic renal cell carcinoma (RCC).

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

As used herein, the term “sample” to any biological sample obtained from the purpose of evaluation in vitro.

In some embodiments, the biological sample is a tissue sample. The term “tissue sample” includes sections of tissues such as biopsy or autopsy samples and frozen sections taken for histological purposes. In some embodiments, the tissue sample may result from a biopsy performed in the RCC of the patient.

In some embodiments, the biological sample is a body fluid sample. Examples of body fluids are blood, serum, plasma, amniotic fluid, brain/spinal cord fluid, liquor, cerebrospinal fluid, sputum, throat and pharynx secretions and other mucous membrane secretions, synovial fluids, ascites, tear fluid, lymph fluid and urine. More particularly, the sample is a blood sample. As used herein, the term “blood sample” refers to a whole blood sample, serum sample and plasma sample. A blood sample may be obtained by methods known in the art including venipuncture or a finger stick. Serum and plasma samples may be obtained by centrifugation methods known in the art. The sample may be diluted with a suitable buffer before conducting the assay.

As used herein, the term “IL-34” has its general meaning in the art and refers to the interleukin-34 that is characterized by the amino acid sequence as set forth in SEQ ID NO:1.

>sp|Q6ZMJ4|IL34_HUMAN Interleukin-34 OS = Homo
sapiens OX = 9606 GN = IL34 PE = 1 SV = 1
SEQ ID NO: 1
MPRGFTWLRYLGIFLGVALGNEPLEMWPLIQNEECTVIGFLRDKLQYRS
RLQYMKHYFPINYKISVPYEGVFRIANVIRLQRAQVSERELRYLWVLVS
LSATESVQDVLLEGHPSWKYLQEVEILLLNVQQGLIDVEVSPKVESVLS
LLNAPGPNLKLVRPKALLDNCFRVMELLYCSCCKQSSVLNWQDCEVPSP
QSCSPEPSLQYAATQLYPPPPWSPSSPPHSTGSVRPVRAQGEGLLP

As used herein, the term “SAA2” has its general meaning in the art and refers to the Serum amyloid A-2 protein that is characterized by the amino acid sequence as set forth in SEQ ID NO:2.

>sp|P0DJI9|SAA2_HUMAN Serum amyloid A-2 protein
OS = Homo sapiens OX = 9606 GN = SAA2 PE = 1
SV = 1
SEQ ID NO: 2
MKLLTGLVFCSLVLSVSSRSFFSFLGEAFDGARDMWRAYSDMREANYIG
SDKYFHARGNYDAAKRGPGGAWAAEVISNARENIQRLIGRGAEDSLADQ
AANKWGRSGRDPNHFRPAGLPEKY

As used herein, the term “PONL1” has its general meaning in the art and refers to the Podocan-like protein 1 that is characterized by the amino acid sequence as set forth in SEQ ID NO:3.

>sp|Q6PEZ8|PONL1_HUMAN Podocan-like protein 1
OS = Homo sapiens OX = 9606 GN = PODNL1 PE = 2
SV = 2
SEQ ID NO: 3
MAESGLAMWPSLLLLLLLPGPPPVAGLEDAAFPHLGESLQPLPRACPLR
CSCPRVDTVDCDGLDLRVFPDNITRAAQHLSLQNNQLQELPYNELSRLS
GLRTLNLHNNLISSEGLPDEAFESLTQLQHLCVAHNKLSVAPQFLPRSL
RVADLAANQVMEIFPLTFGEKPALRSVYLHNNQLSNAGLPPDAFRGSEA
TATLSLSNNQLSYLPPSLPPSLERLHLQNNLISKVPRGALSRQTQLREL
YLQHNQLTDSGLDATTFSKLHSLEYLDLSHNQLTTVPAGLPRTLAILHL
GRNRIRQVEAARLHGARGLRYLLLQHNQLGSSGLPAGALRPLRGLHTLH
LYGNGLDRVPPALPRRLRALVLPHNHVAALGARDLVATPGLTELNLAYN
RLASARVHHRAFRRLRALRSLDLAGNQLTRLPMGLPTGLRTLQLQRNQL
RMLEPEPLAGLDQLRELSLAHNRLRVGDIGPGTWHELQALQVRHRLVSH
TVPRAPPSPCLPCHVPNILVSW

As used herein, the term “CFB” has its general meaning in the art and refers to the Complement factor B that is that is characterized by the amino acid sequence as set forth in SEQ ID NO:4.

>sp|P00751|CFAB_HUMAN Complement factor B OS =
Homo sapiens OX = 9606 GN = CFB PE = 1 SV = 2
SEQ ID NO: 4
MGSNLSPQLCLMPFILGLLSGGVITTPWSLARPQGSCSLEGVEIKGGSF
RLLQEGQALEYVCPSGFYPYPVQTRICRSIGSWSTLKTQDQKTVRKAEC
RAIHCPRPHDFENGEYWPRSPYYNVSDEISFHCYDGYILRGSANRICQV
NGRWSGQTAICDNGAGYCSNPGIPIGIRKVGSQYRLEDSVTYHCSRGLT
LRGSQRRTCQEGGSWSGTEPSCQDSFMYDTPQEVAEAFLSSLTETIEGV
DAEDGHGPGEQQKRKIVLDPSGSMNIYLVLDGSDSIGASNFTGAKKCLV
NLIEKVASYGVKPRYGLVIYATYPKIWVKVSEADSSNADWVIKQLNEIN
YEDHKLKSGTNIKKALQAVYSMMSWPDDVPPEGWNRTRHVIILMIDGLH
NMGGDPITVIDEIRDLLYIGKDRKNPREDYLDVYVFGVGPLVNQVNINA
LASKKDNEQHVFKVKDMENLEDVFYQMIDESQSLSLCGMVWEHRKGIDY
HKQPWQAKISVIRPSKGHESCMGAVVSEYFVLTAAHCFTVDDKEHSIKV
SVGGEKRDLEIEVVLFHPNYNINGKKEAGIPEFYDYDVALIKLKNKLKY
GQIIRPICLPCTEGTTRALRLPPITTCQQQKEELLPAQDIKALFVSEEE
KKLTRKEVYIKNGDKKGSCERDAQYAPGYDKVKDISEVVIPRFLCIGGV
SPYADPNICRGDSGGPLIVHKRSRFIQVGVISWGVVDVCKNQKRQKQVP
AHARDFHINLFQVLPWLKEKLQDEDLGFL

The measurement of the level of biomarker in the sample, in particular in the blood sample, is typically carried out using standard protocols known in the art.

For example, the method may comprise contacting the blood sample with a binding partner capable of selectively interacting with the biomarker in the sample. In some embodiments, the binding partners are antibodies, such as, for example, monoclonal antibodies or even aptamers. For example the binding may be detected through use of a competitive immunoassay, a non-competitive assay system using techniques such as western blots, a radioimmunoassay, an ELISA (enzyme linked immunosorbent assay), a “sandwich” immunoassay, an immunoprecipitation assay, a precipitin reaction, a gel diffusion precipitin reaction, an immunodiffusion assay, an agglutination assay, a complement fixation assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, and a BIAcore analysis. The aforementioned assays generally involve the binding of the partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. An exemplary biochemical test for identifying specific proteins employs a standardized test format, such as ELISA test, although the information provided herein may apply to the development of other biochemical or diagnostic tests and is not limited to the development of an ELISA test (see, e.g., Molecular Immunology: A Textbook, edited by Atassi et al. Marcel Dekker Inc., New York and Basel 1984, for a description of ELISA tests). Therefore ELISA method can be used, wherein the wells of a microtiter plate are coated with a set of antibodies which recognize the biomarker. A sample containing or suspected of containing the biomarker is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art. Measuring the level of the biomarker (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e.g., retention time, for that compound and measured using standard techniques. Alternatively, the separated compounds may be detected and measured by, for example, a mass spectrometer. Typically, levels of immunoreactive the biomarker in a sample may be measured by an immunometric assay on the basis of a double-antibody “sandwich” technique, with a monoclonal antibody specific for the biomarker (Cayman Chemical Company, Ann Arbor, Mich.). According to said embodiment, said means for measuring the biomarker level are for example i) a the biomarker buffer, ii) a monoclonal antibody that interacts specifically with the biomarker, iii) an enzyme-conjugated antibody specific for the biomarker and a predetermined reference value of the biomarker.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of the biomarker in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the biomarker in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples; b) providing, for each ample provided at step a), information relating to the actual clinical outcome for the corresponding subject (i.e. the duration of the survival); c) providing a serial of arbitrary quantification values; d) determining the expression level of the biomarker for each sample contained in the collection provided at step a); e) classifying said samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the patients from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example the expression level of the biomarker has been assessed for 100 samples of 100 patients. The 100 samples are ranked according to the expression level of the biomarker. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of the biomarker corresponding to the boundary between both subsets for which the p value is minimum is considered as the predetermined reference value.

Typically, an expression level of the biomarker that is higher than the predetermined reference value indicates that the patient will have a short survival time and an expression level that is higher than the predetermined reference value indicates that the patient will have a long survival time.

It should be noted that the predetermined reference value is not necessarily the median value of expression levels of the gene. Thus in some embodiments, the predetermined reference value thus allows discrimination between a poor and a good prognosis for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of the biomarker with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the expression level of the biomarker, where values higher than 5 reveal a poor prognosis and values less than 5 reveal a good prognosis. In some embodiments, a patient may be assessed by comparing values obtained by measuring the expression level of the biomarker and comparing the values on a scale, where values above the range of 4-6 indicate a poor prognosis and values below the range of 4-6 indicate a good prognosis, with values falling within the range of 4-6 indicating an intermediate occurrence (or prognosis).

In some embodiments, the expression levels of 2 biomarkers are determined in the sample. In some embodiments, the expression levels of 3 biomarkers are determined in the sample. In some embodiments, the expression levels of the 4 biomarkers (IL-34, SAA2, PONL1 and CFB) are determined in the sample.

In some embodiments, a score which is a composite of the expression levels of the different biomarkers is determined and compared to the predetermined reference value wherein a difference between said score and said predetermined reference value is indicative whether the patient will have a long or short survival time.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF) such as described in the Example. In some embodiments, the method of the invention comprises the step of determining the patient response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n patients to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of a plurality of biomarkers in the sample; b) implementing a classification algorithm on data comprising the quantified plurality of biomarkers so as to obtain an algorithm output; c) determining the prognosis from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiment, in view of the currently limited options for RCC management, the group of biomarkers as disclosed herein is useful for identifying patients with poor-prognosis, in particular patients with localized RCCs that are likely to relapse and metastasize. Accordingly, subject identified with a poor prognosis can be administered therapy, for example systematic therapy. In some embodiments, the method of the present invention be used to identify patients in need of frequent follow-up by a physician or clinician to monitor RCC disease progression. Screening patients for identifying patients having a poor prognosis using the group of the biomarkers as disclosed herein is also useful to identify patients most suitable or amenable to be enrolled in clinical trial for assessing a therapy for RCC, which will permit more effective subgroup analyses and follow-up studies. Furthermore, the expression of the group of biomarkers as disclosed herein can be monitored in patients enrolled in a clinical trial to provide a quantitative measure for the therapeutic efficacy of the therapy which is subject to the clinical trial.

This invention also provides a method for selecting a therapeutic regimen or determining if a certain therapeutic regimen is more appropriate for a patient identified as having a poor prognosis as identified by the methods as disclosed herein. For example, an aggressive anti-cancer therapeutic regime can be perused in which a patient having a poor prognosis, where the patient is administered a therapeutically effective amount of an anti-cancer agent to treat the RCC. In some embodiments, a patient can be monitored for RCC using the methods and biomarkers as disclosed herein, and if on a first (i.e. initial) testing the patient is identified as having a poor prognosis, the patient can be administered an anti-cancer therapy, and on a second (i.e. follow-up testing), the patient is identified as having a good prognosis, the patient can be administered an anti-cancer therapy at a maintenance dose. The method of the present invention is particularly suited to determining which patients will be responsive or experience a positive treatment outcome to a treatment.

In general, a therapy is considered to “treat” RCC if it provides one or more of the following treatment outcomes: reduce or delay recurrence of the RCC after the initial therapy; increase median survival time or decrease metastases. In some embodiments, an anti-cancer therapy is, for example but not limited to administration of a chemotherapeutic agent, radiotherapy etc. Such anti-cancer therapies are disclosed herein, as well as others that are well known by persons of ordinary skill in the art and are encompassed for use in the present invention. The term “anti-cancer agent” or “anti-cancer drug” is any agent, compound or entity that would be capably of negatively affecting the cancer in the patient, for example killing cancer cells, inducing apoptosis in cancer cells, reducing the growth rate of cancer cells, reducing the number of metastatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the patient with cancer. Anti-cancer therapy includes biological agents (biotherapy), chemotherapy agents, and radiotherapy agents. In some embodiments, the anti-cancer therapy includes a chemotherapeutic regimen further comprises radiation therapy. In some embodiments, the anti-cancer treatment comprises the administration of a chemotherapeutic drug, alone or in combination with surgical resection of the tumor. In some embodiments, the treatment compresses radiation therapy and/or surgical resection of the tumor masses.

The term “chemotherapeutic agent” or “chemotherapy agent” are used interchangeably herein and refers to an agent that can be used in the treatment of cancers and neoplasms. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex or Trexall), irinotecan (CPT-11); erlotinib; oxalipatin; anthracyclins-idarubicin and daunorubicin; doxorubicin; alkylating agents such as melphalan and chlorambucil; cis-platinum, methotrexate, and alkaloids such as vindesine and vinblastine.

In some embodiments, the patients are administered with anti-VEGF agents. As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof. In some embodiments, the anti-VEGF agent is Sunitinib (marketed as Sutent by Pfizer, and previously known as SU11248) that is an oral, small-molecule, multi-targeted receptor tyrosine kinase (RTK) inhibitor that was approved by the FDA for the treatment of renal cell carcinoma (RCC).

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The invention also provides diagnostic and experimental kits which include antibodies for determining the protein expression level encoded by at least 2 or at least 3 biomarkers as disclosed herein, in order to determine the prognosis of the patient suffering from cancer. In such kits, the antibodies may be provided with means for binding to detectable marker moieties or substrate surfaces. Alternatively, the kits may include the antibodies already bound to marker moieties or substrates. The kits may further include reference biological samples as well as positive and/or negative control reagents as well as other reagents for adapting the use of the antibodies to particular experimental and/or diagnostic techniques as desired. The kits may be prepared for in vivo or in vitro use, and may be particularly adapted for performance of any of the methods of the invention, such as ELISA. For example, kits containing antibody bound to multi-well microtiter plates can be manufactured.

Methods of Treatment:

A further object of the present invention relates to a method of treating renal cell carcinoma (RCC) in a patient in need thereof comprising administering to the subject a therapeutically effective amount of an inhibitor of IL-34, SAA2, PONL1 or CFB.

In some embodiments, the patient was previously predicted as having a poor prognosis by the method of the present invention.

As used herein, the term “inhibitor” refers to a compound, substance or composition that can inhibit the function and/or expression of the targeted protein (i.e. IL-34, SAA2, PONL1 or CFB). For example, the inhibitor can inhibit the expression or activity of the protein, modulate or block the protein binding to its receptor or ligand or block the signalling pathway that results from the activation of the protein. In particular, the inhibitor inhibits the interaction between targeted protein (i.e. IL-34, SAA2, PONL1 or CFB) and its partners (receptor or ligand). Typically, the inhibitor is a small organic molecule, a nucleic acid, or a protein such as an antibody.

In some embodiments, the inhibitor is an antibody having specificity for IL-34, SAA2, PONL1 or CFB.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In some embodiments, the antibody is a single domain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”.

In some embodiments, the antibody is a chimeric antibody. As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

In some embodiments, the antibody is a humanized antibody. In particular, in said humanized antibody, the variable domain comprises human acceptor frameworks regions, and optionally human constant domain where present, and non-human donor CDRs, such as mouse CDRs. According to the invention, the term “humanized antibody” refers to an antibody having variable region framework and constant regions from a human antibody but retains the CDRs of a previous non-human antibody. In one embodiment, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

In some embodiments, the antibody is a human antibody. As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

In some embodiments, the inhibitor is an inhibitor of expression. An “inhibitor of expression” refers to a natural or synthetic compound that has a biological effect to inhibit the expression of a gene that encodes for e.g. IL-34, SAA2, PONL1 or CFB.

In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of targeted protein (i.e. IL-34, SAA2, PONL1 or CFB) mRNA by binding thereto and thus preventing protein translation or increasing mRNA degradation, thus decreasing the level of targeted protein (i.e. IL-34, SAA2, PONL1 or CFB), and thus activity, in a cell. For example, antisense oligonucleotides of at least about 15 bases and complementary to unique regions of the mRNA transcript sequence encoding targeted protein (i.e. IL-34, SAA2, PONL1 or CFB) can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. targeted protein (i.e. IL-34, SAA2, PONL1 or CFB) gene expression can be reduced by contacting a patient or cell with a small double stranded RNA (dsRNA), or a vector or construct causing the production of a small double stranded RNA, such that targeted protein (i.e. IL-34, SAA2, PONL1 or CFB) gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing targeted protein (i.e. IL-34, SAA2, PONL1 or CFB). Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of expression is an endonuclease.

The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). The DNA targeting endonuclease can be a naturally occurring endonuclease (e.g., a bacterial meganuclease) or it can be artificially generated (e.g., engineered meganucleases, TALENs, or ZFNs, among others).

In some embodiments, the DNA targeting endonuclease of the present invention is a TALEN. As used herein, the term “TALEN” has its general meaning in the art and refers to a transcription activator-like effector nuclease, an artificial nuclease which can be used to edit a target gene. TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29: 149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29: 135-6; and Boch et al. (2009) Science 326: 1509-12; Moscou et al. (2009) Science 326: 3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29: 149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96). The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29: 143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17: 11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the DNA targeting endonuclease of the present invention is a ZFN. As used herein, the term “ZFN” or “Zinc Finger Nuclease” has its general meaning in the art and refers to a zinc finger nuclease, an artificial nuclease which can be used to edit a target gene. Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188: 773-782; and Kim et al. (1996) Proc. Natl. Acad. Sci. USA 93: 1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95: 10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the DNA targeting endonuclease of the present invention is a CRISPR-associated endonuclease. As used herein, the term “CRISPR-associated endonuclease” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, Mass.).

In some embodiments, the CRISPR-associated endonuclease is a Cpf1 nuclease. As used herein, the term “Cpf1 protein” to a Cpf1 wild-type protein derived from Type V CRISPR-Cpf1 systems, modifications of Cpf1 proteins, variants of Cpf1 proteins, Cpf1 orthologs, and combinations thereof. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532(7600):517-21 (2016)).

As used herein, the term “therapeutically effective amount” refers to an amount effective, at dosages and for periods of time necessary, to achieve a desired therapeutic result. A therapeutically effective amount of the inhibitor may vary according to factors such as the disease state, age, sex, and weight of the individual, and the ability of the inhibitor to elicit a desired response in the individual. A therapeutically effective amount is also one in which any toxic or detrimental effects of the antibody or antibody portion are outweighed by the therapeutically beneficial effects. The efficient dosages and dosage regimens for the inhibitor depend on the disease or condition to be treated and may be determined by the persons skilled in the art. A physician having ordinary skill in the art may readily determine and prescribe the effective amount of the pharmaceutical composition required. For example, the physician could start doses of inhibitor employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of an inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of an inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

According to the present invention, the inhibitor is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Interleukin-34 (IL34) expression in mouse and human samples. (A) IL34 expression increases in mouse cell lines rendered increasingly aggressive by in vivo passages. (B) IL34 protein secretion in conditioned media of Passage 6 cell lines (Kidney, Lung and Tail). (c-d) High versus Low IL34 expression predicts Overall (C) and Progression-Free (D) survival in TCGA ccRCC patient cohort. (E-G) In UroCCR patient tissue samples, IL34 RNA is overexpressed in tumour versus healthy kidney (e), increases with Fuhrman grade (F) and correlates with reduced overall patient survival. (H-I) IL34 staining score correlates with Fuhrman Grade (i) and is predictive of Progression Free Survival (j).

FIG. 2 IL34 Crispr-Cas9 deletion and response to Sutent treatment. (A) IL34 deletion via three different Crispr-Cas9 constructs (CrisprIL34-1a, 1b, 1c) in RENCA cells strongly inhibits primary tumour formation versus control (Crispr-LacZ), leading to reduced tumour weight. (B-C) IL34 deletion via three different Crispr-Cas9 constructs in RENCA cells strongly inhibits experimental metastasis formation (Tail Vein injection, b). Metastases account for a reduced % of lung tissue area (B) and have reduced numbers of MMR+ cells (type 2 macrophages, C). (D) Plasma IL34 levels rise markedly in a subset of patients given first-cycle Sutent therapy for metastases. (E) In a mouse xenograft model, subcutaneous tumours from mice treated with Sutent (sunitinib) versus vehicle control (CT) showed increased levels of IL34 RNA. Species specific qPCR primers show upregulation in both human (tumour cell) and mouse (stromal cell) compartments (E-F).

FIG. 3. Serum Amyloid Protein A2 (SAA2) expression in mouse and human RCC samples. ( ) SAA2 mRNA expression is upregulated with passage in Lung cell lines (transcriptomic data). (B-C) High versus Low SAA2 expression predicts Overall (b) and Progression-Free (c) survival in TCGA ccRCC cohort. (D-E) SAA2 mRNA is highly expressed in both healthy kidney and tumour tissue (D) however expression in tumour samples increases with grade (E) in UroCCR patient tissue samples. (F-G) mRNA expression is predictive of shortened Overall (F) and Progression Free (G) survival in UroCCR patient cohort.

FIG. 4. Serum Amyloid Protein A2 (SAA2) in patient plasma/serum samples. (A-C) UroCCR patients, plasma collected from 47 patients prior to surgical resection of primary tumour. Mean plasma SAA2 level is increased in patients with concomitant metastases (M1) or patients without metastases who later develop them (M0 progressors) versus patients without metastases who do not progress during follow-up (M0 non progressors). Higher plasma level predicts shortened Progression Free (B) and Overall (C) survival. (D-F) SUVEGIL 11 patients, following surgical resection of primary tumour. Serum samples collected at diagnosis with metastases, and following one cycle of Sutent treatment. Patients who did not show progression following treatment tended to show a decrease in SAA2 level following treatment (D), whereas patients who's metastases progressed (nonresponders) showed increased levels (E, F).

FIG. 5. Complement Factor B (CFb) expression in mouse and human RCC samples. (A) CFb mRNA expression is upregulated with passage in Lung and Tail cell lines (qPCR data). (B) CFb mRNA expression is increased in RENCA isolated from metastases versus their primary tumours of origin (paired samples). (c, d) High versus Low CFb expression predicts Overall (C) and Progression-Free (D) survival in TCGA ccRCC cohort. (E-G) CFb mRNA is overexpressed in tumour versus healthy UroCCR tissue samples, and predicts shortened Overall (F) and Progression-Free (G) survival.

FIG. 6. Complement Factor B (CFb) in patient plasma/serum samples. (A) UroCCR patients, plasma collected from 47 patients prior to surgical resection of primary tumour. Mean plasma CFb level is increased in patients with concomitant metastases (M1) or who patients without metastases who later progress (M0 progressors) versus patients without metastases who do not progress during followup (M0 non progressors). (B) UroCCR patients. Plasma collected from N=20 patients approximately 1 month following surgery for primary tumour removal. The plasma CFb level is again higher in patients with metastases (M1) versus those without (M0). (C) SUVEGIL 11 patients, following surgical resection of primary tumour. Serum samples collected at diagnosis with metastases, and following one cycle of Sutent treatment. Patients who's CFb serum level increased following treatment had faster progression than those who's level decreased. (D) SUVEGIL 11 patients, divided into three groups according to increase/decrease in both SAA2 and CFb as combinatorial analysis. Patients with decrease in neither protein show best outcome, and the patient with increase in both the worst outcome.

FIG. 7 Podocan-like1 (Podnl1) expression in mouse and human RCC samples. (A, B) Podnl mRNA expression is upregulated with passage in Kidney cell lines (microarray, b qPCR data). (C) Podnl mRNA expression is increased in RENCA isolated from primary tumours versus in vitro culture but is not further increased in metastatic tumour cells (paired samples). (D, E) High versus Low Podnl1 expression predicts Overall (D) and Progression-Free (E) survival in TCGA ccRCC cohort. (F, G) Podnl1 mRNA is overexpressed in tumour versus healthy UroCCR tissue samples, and predicts shortened Overall (F) and Progression-Free (G) survival.

FIG. 8. Clinical relevance of SAA2 and CFB after anti-angiogenic treatment (SUVEGILTORAVA cohorts). (A and B) Correlation between plasmatic SAA2 levels at diagnosis and survival (OS and PFS) in patients after sunitinib treatment (plasmatic level at the diagnosis less or greater than a cut-off for SAA2 (269 μg/ml) [OS: HR(log-rank)=5.557; PFS: HR(logrank)=7.669. (C) Correlation between plasmatic SAA2 levels at diagnosis and PFS in patients after sunitinib or bevacizumab treatment (plasmatic level at the diagnosis less or greater than a third quartile cut-off for SAA2 (269 μg/ml; HR(log-rank)=1.987). (D) Correlation between plasmatic CFB levels at diagnosis and PFS in patients after sunitinib or bevacizumab treatment (plasmatic level at the diagnosis less or greater than a third quartile cut-off for CFB (310 μg/ml; HR(log-rank)=3.113) (E and F) PFS (E) and OS (F) patients treated with either Sunitinib of bevacizumab and stratified according to plasma levels of both SAA2 and CFB. Three subgroups were identified i) CFB low and SAA2 low, ii) CFB low and SAA2 high or CFB high and SAA2 low, iii) CFB high and SAA2 high (Low-low vs high-high: OS HR(log-rank)=5.086; PFS HR(log-rank)=4.196).

### EXAMPLE

Methods

Mice and Cell Lines

Female BALB/c mice 6-8 weeks of age were purchased from Charles River Laboratories. Mice were housed in the animal facility of Bordeaux University (Animalerie Mutualisée Bordeaux, France). The GFP expressing Renca murine renal cancer cell line (RENCA-GFP) and sub-cell lines generated (Kidney, Tail, Lung) were maintained in Roswell Park Memorial Institute (RPMI) 1640 medium supplemented with 10% foetal bovine serum (FBS) and 1% penicillin/streptomycin and were incubated at 37° C., 5% CO₂ in an incubator. Crispr-Cas9_IL34 and CrisprCas9_LacZ cell lines were generated using standard protocols.

Mouse Orthtotopic Subcapsular and Experimental Metastasis (Tail Vein Injection) Models.

Tumours were implanted by sub-capsular injections of 1×10⁵ RENCA-GFP cells into the left kidney of wild type BALB/c mice. For the intravenous injections, 5×10⁵ RENCA-GFP cells were injected into the caudal vein of wild type BALB/c mice. When the endpoints defined by the approved protocols were reached, mice were sacrificed, and tumour tissues and lungs were collected. For immunochemistry, tissue were fixed in paraformaldehyde 4% (PFA 4%, Santa Cruz Biotechnology, sc-281692) for 2 hours and then incubated for 72 hours in 30% sucrose. Tissues were frozen in OCT Compound (Tissue-Tek OCT compound, Sakura, 4583). Prior to embedding, lungs were inflated with 1 mL of diluted OCT (1:1 PBS/OCT dilution). Frozen tissues were preserved at −80° C. For protein, DNA and RNA analysis, tissues were snap-frozen in liquid nitrogen and preserved at −80° C.

Tissue Dissociation and Tumour Cell Purification

For tumour cell purification, tissues were cut into small pieces with a scalpel and digested with Collagenase I and Collagenase II (Liberase TL, Roche, 05401020001) for 1 hour at 37° C. To further improve the dissociation, digested tissues were filtered in cell strainers (100 μm, 70 μm and 40 μm) and seeded in complete medium, and incubated at 37° C., 5% CO₂. Cell cultures were checked daily and passaged as necessary. Tumour cell outgrowth and primary cell death resulted in tumour cell only cultures, verified by visualisation of GFP using fluorescence microscopy When no GFP-negative cells could be visually detected, cell cultures were considered sufficiently pure. RENCA-GFP cells were collected for analysis or re-implanted into mice for further in-vivo passage. In some cases, cells were cultured in serum free media for 24 hrs to generate conditioned medium.

Xenograft mouse experiments were done with subcaneously injected 786-0 human RCC cells in immunodeficient mice and treated with sunitnib (40 mg/kg) according to published protocols (Dufies et al, Cancer Res, March 2017 DOI: 10.1158/0008-5472.CAN-16-3088)

Gene Expression Analysis

Total RNA was extracted using the RNeasy Plus Mini Kit (Qiagen, #74134), according to the manufacturer's instructions. Agilent mouse full Genomic Array was used for transcriptomic analysis.

Quantitative PCR (qPCR) analyses: 1 μg of total RNA was reverse-transcribed into complementary DNA (cDNA) using the high-capacity cDNA reverse transcription kit (Applied Biosystems, 4368814). The resulting cDNA were amplified using specific primers for the genes of interest. HPRT was used as internal control.

Enzyme-linked immunosorbent assays (ELISA) were performed according to the manufacturer's instructions on conditioned media or human plasma or serum samples.

Patient Samples

UroCCR Tissue Bank.

Clinical data and biological samples (frozen/paraffin-embedded tissue, plasma and urine samples) were obtained from the French research network on kidney cancer www.uroCCR.fr funded by INCa and localised in Bordeaux. ClinicalTrials.gov identifier: NCT03293563. These samples are referred to as UroCCR cohort. Tissue samples were obtained from patients on the day of surgery for removal of the primary tumour. Plasma and urine samples were obtained either on the day of surgery for the primary tumour or at a time point approximately one month following surgery.

SUVEGIL Serum Samples.

Serum samples from the SUVEGIL clinical trial (Sunitinib Malate in Treating Patients With Kidney Cancer, ClinicalTrials.gov identifier NCT00943839). Patients receive oral sunitinib malate once daily on days 1-28. Courses repeat every 6 weeks in the absence of disease progression or unacceptable toxicity. Blood samples are collected at baseline and then every 6 weeks for pharmacokinetic analysis. In this case, samples tested were obtained at the point of diagnosis of metastases and following the first cycle of treatment.

Immunochemistry and Immunofluorescence

Mouse tissues: For frozen mouse tissues obtained from experiments using CrisprCas9_IL34 and CrisprCas9_LacZ cell lines 10 μm sections were performed with a cryostat (Leica CM1900). For frozen tissue immunofluorescence, sections were incubated 1 hour with a blocking buffer (5% BSA in PBS). Slides were incubated overnight with primary antibody (MMR: R&D Systems, AF2535; GFP: Torrey Pines Biolabs, TP401=>table), and then with secondary fluorescent antibody (REFERENCE=>table) and DAPI (Roche, 10236276001). Images were obtained using a slide scanner (Hamamatsu, Nanozoomer 2.0HT), and processed using NDP.scan software (Hamamatsu). Image analysis using Fiji software (Schindelin, J.; Arganda-Carreras, I. & Frise, E. et al. (2012) Nature methods 9(7): 676-682) was used to calculate the area of tumour tissue as percentage of total tissue section area (%) based on GFP staining. Type 2 macrophage density in tumour tissue was calculated by counting number of MMR-positive cells/pixel area using the “Cell Counter” plugin (Kurt de Vos). Mean areas/cell counts are expressed normalised to those obtained from control tumours.

Human tissues: For paraffined tissues sections were prepared with a microtome. For paraffin tissue sections slides were deparaffinised, re-hydrated and heated in Antigen Retrieval Solution pH6 (HIER Sodium Citrate Buffer, pH6; 10 mM Sodium Citrate, 0.05% Tween 20, pH 6,0). To block endogenous peroxidase activity, slices were treated with 0,3% hydrogen peroxide. After 1 hour of blocking in PBS 5% BSA, slides were incubated overnight with primary antibody (see table), and then incubated with biotinylated secondary antibody for 1 h (see table). Secondary antibodies were HRP-conjugated using the “ABC” technique (Vectastain PK-6100) and then revealed with a peroxidase substrate kit (DAB, Vector Laboratories, SK-4100).

In Silico Analyses

Transcriptional and clinical patient data was obtained from The Human Genome Atlas via the BioPortal website. using the Kidney Renal Cell Carcinoma (KIRC) database. Kaplan Meier graphs representing Overall Survival (OS) and Progression Free Survival (PFS) and all statistical analyses were performed using GraphPad Prism software. For Kaplan Meier analyses, where patient numbers per high/low group are not stated, the cut point is the median value.

Results:

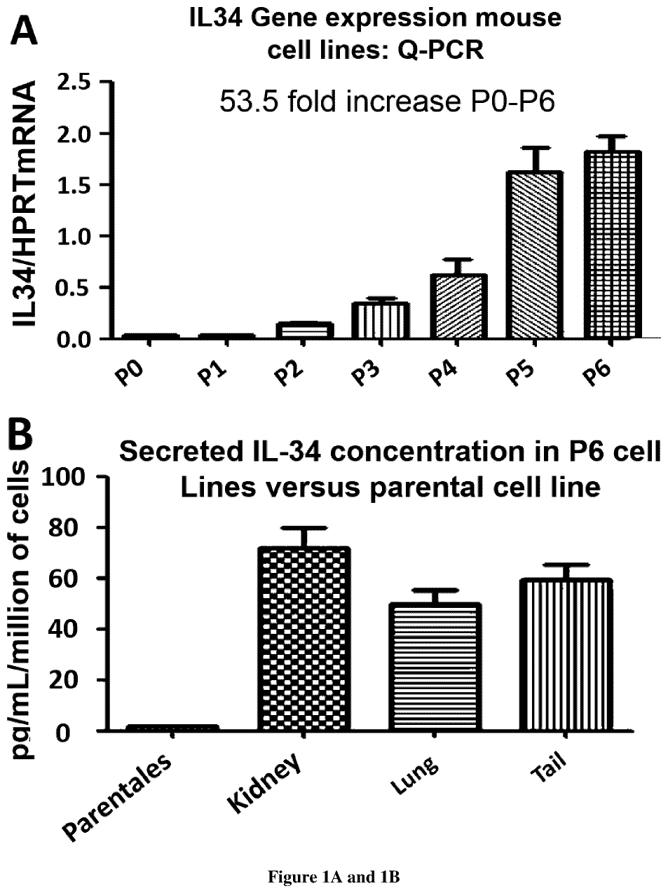

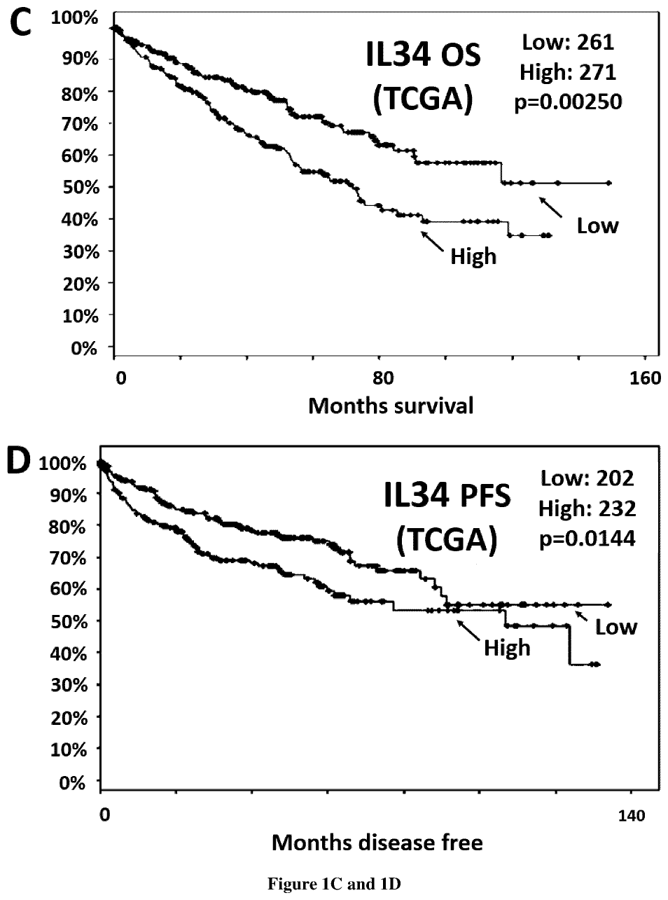

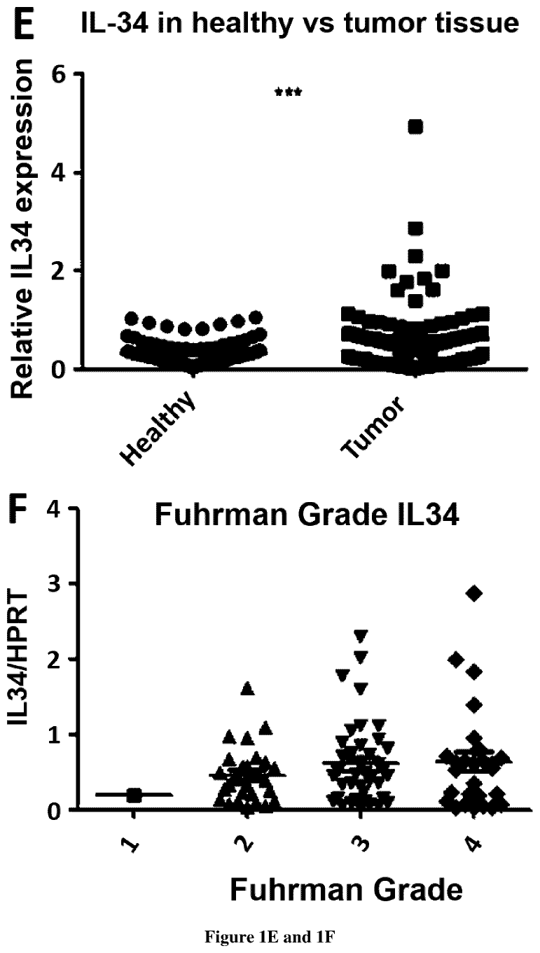

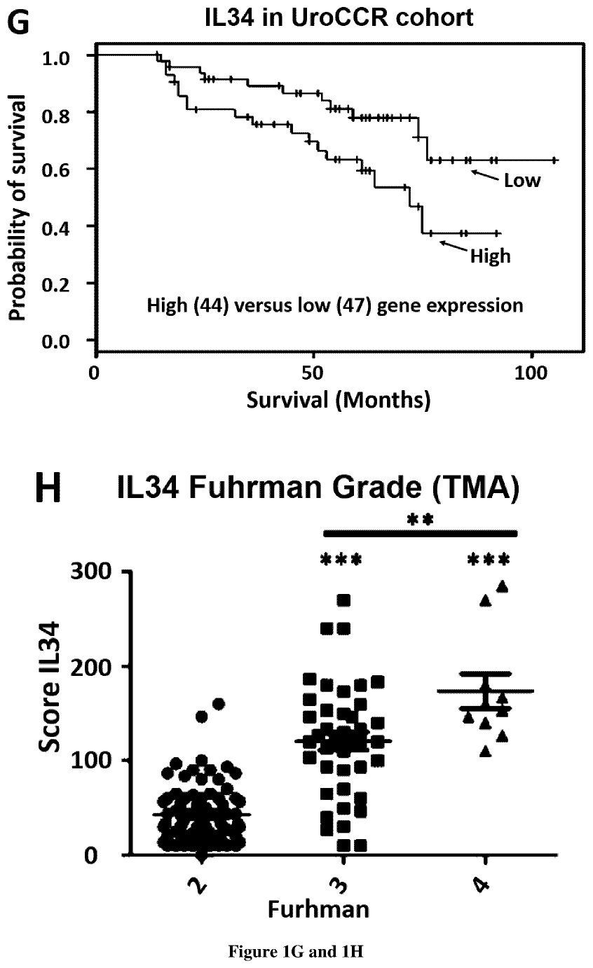

Results are depicted in FIG. 1-7.

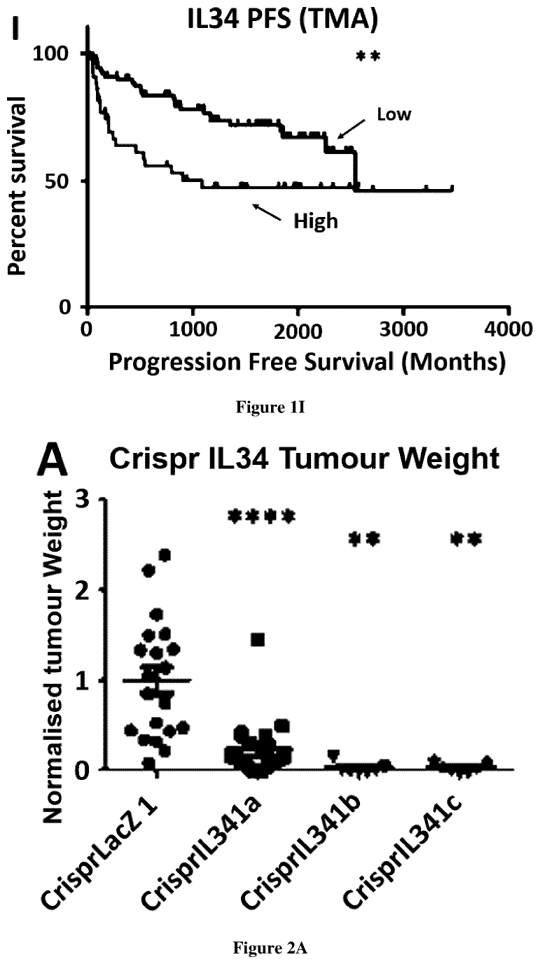

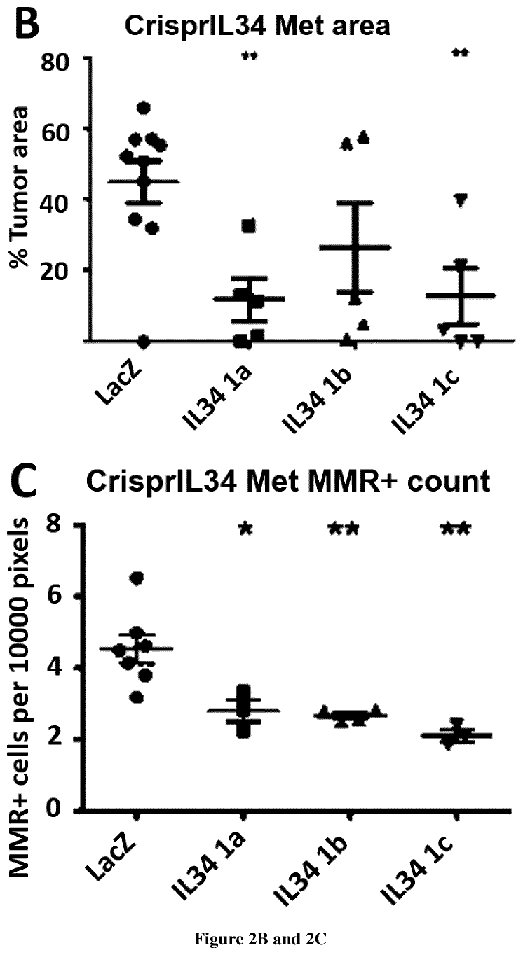

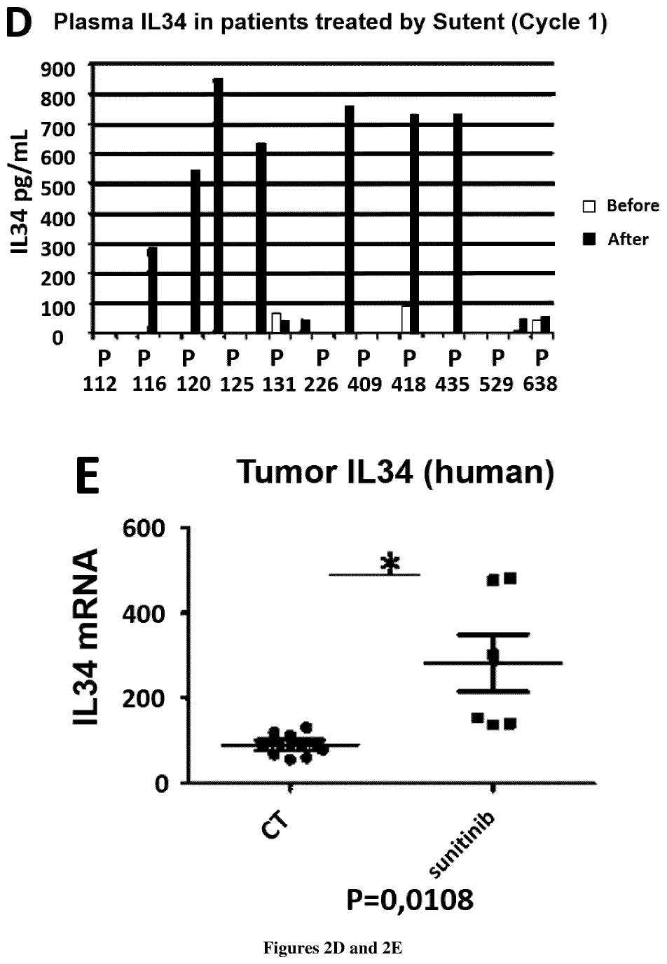

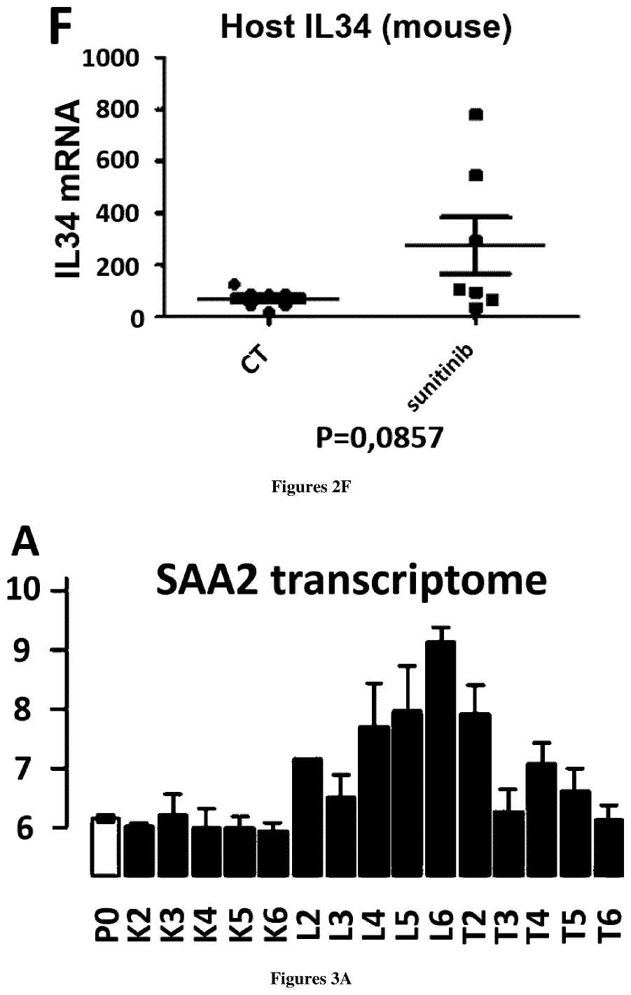

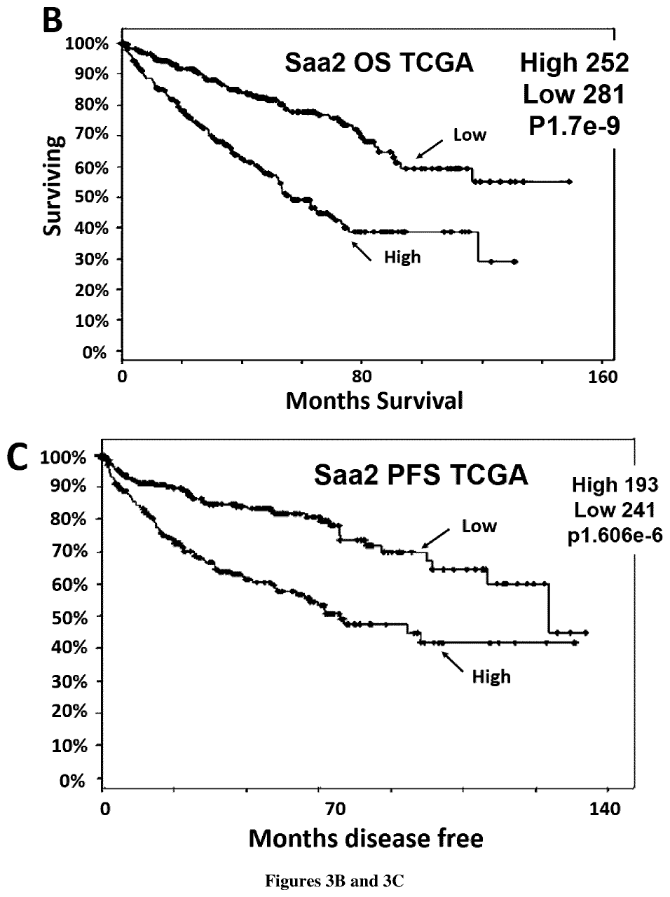

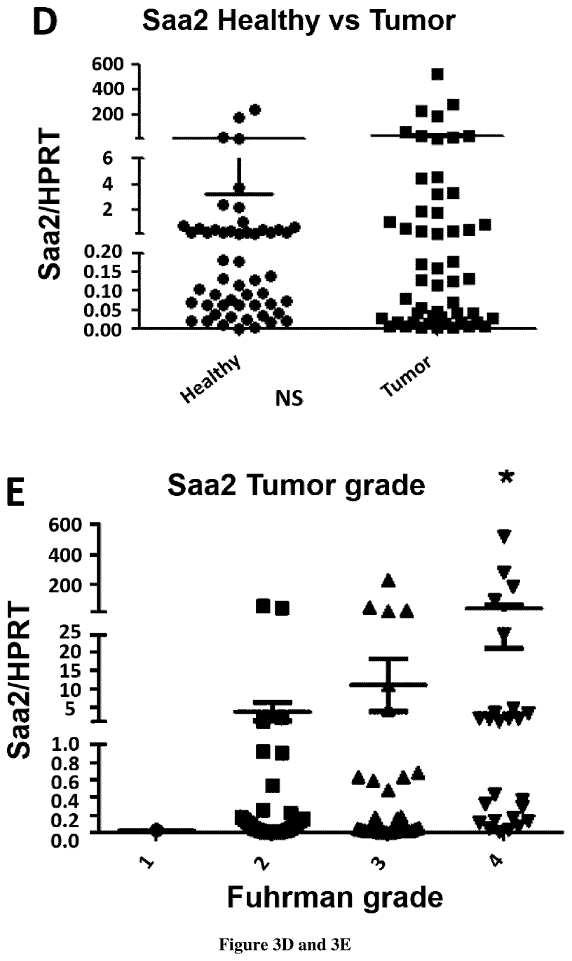

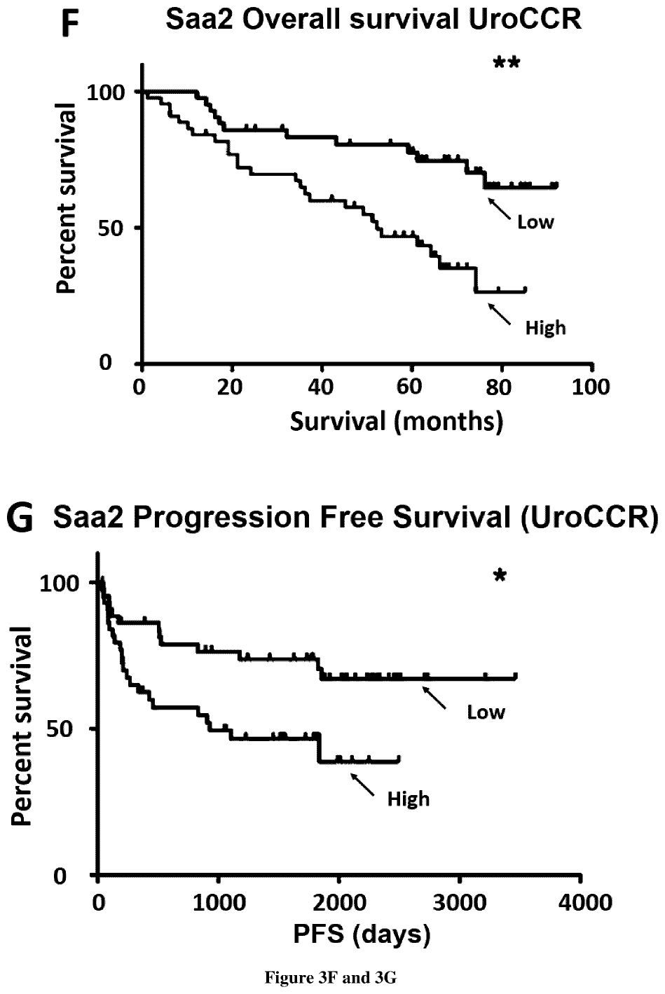

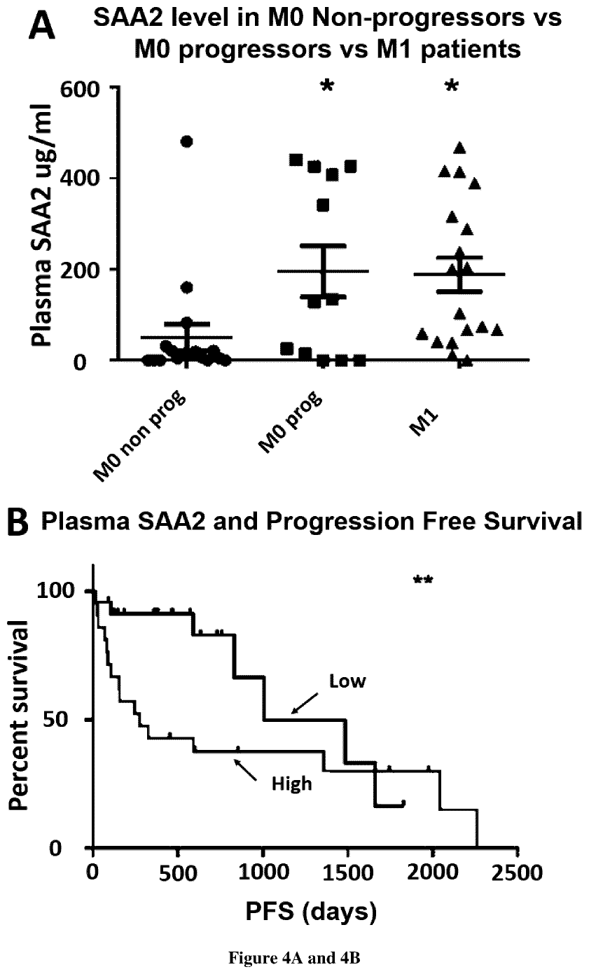

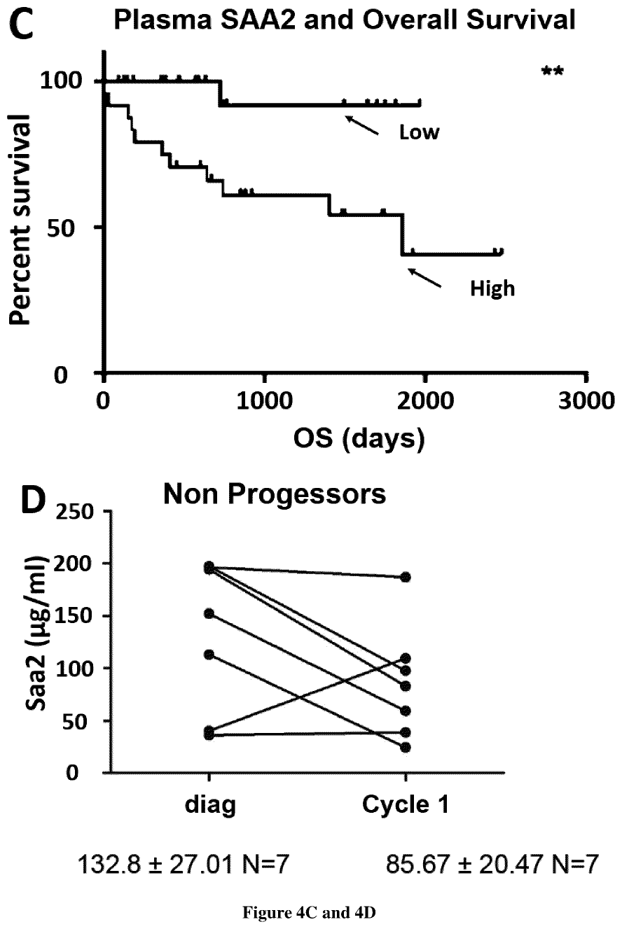

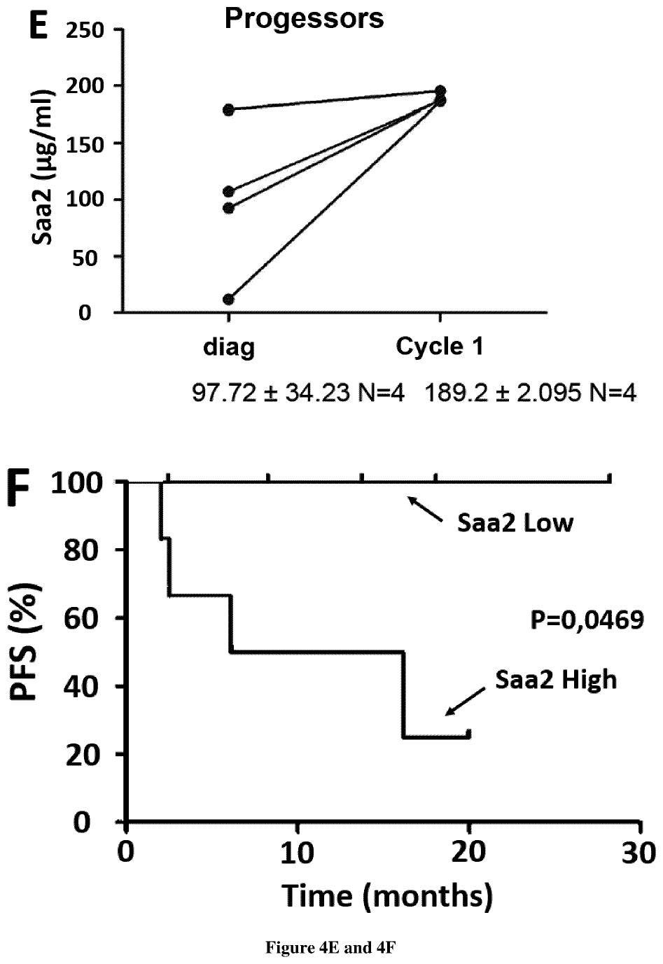

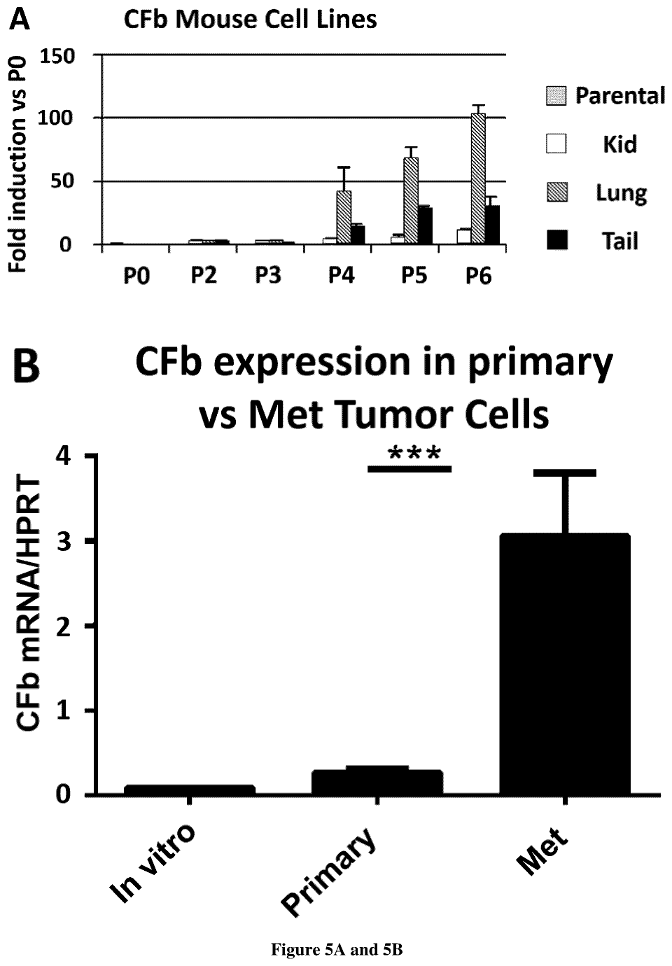

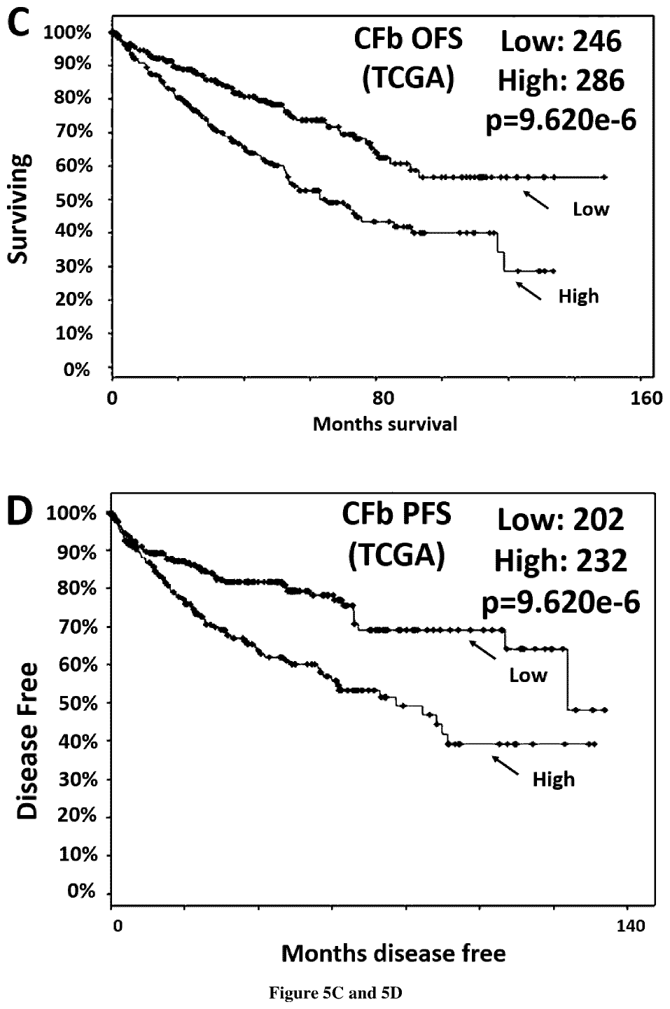

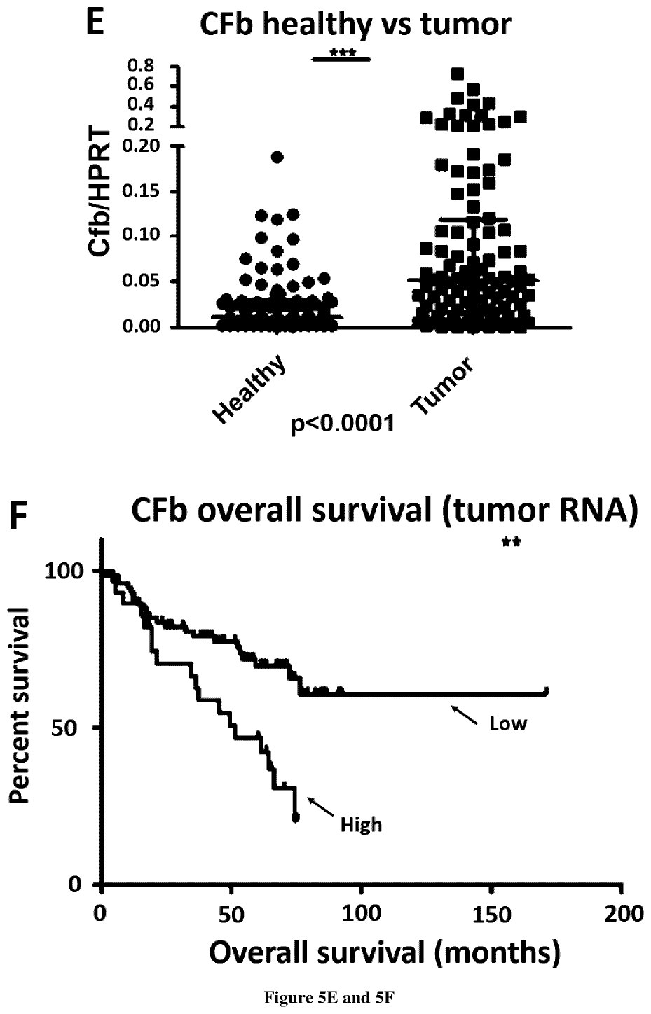

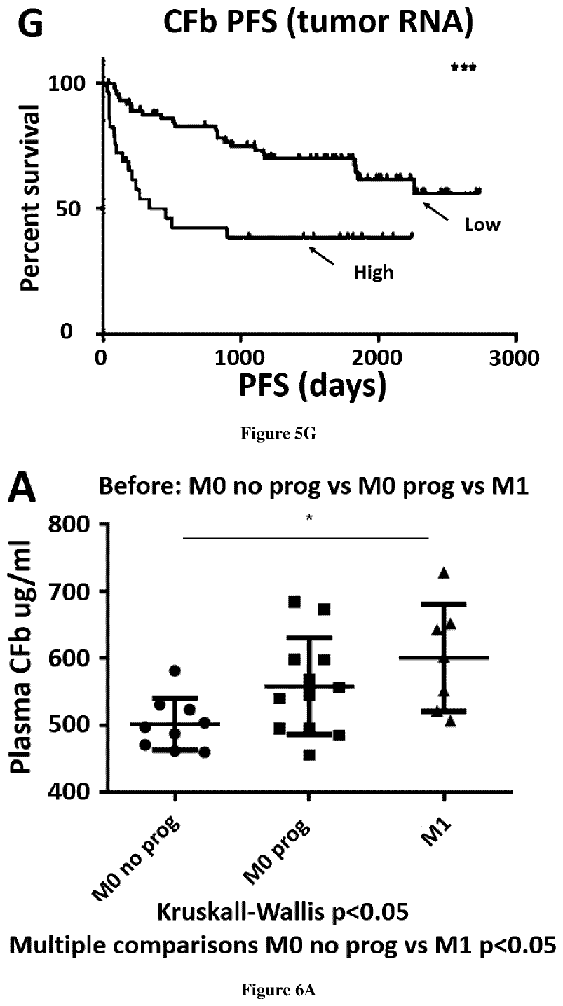

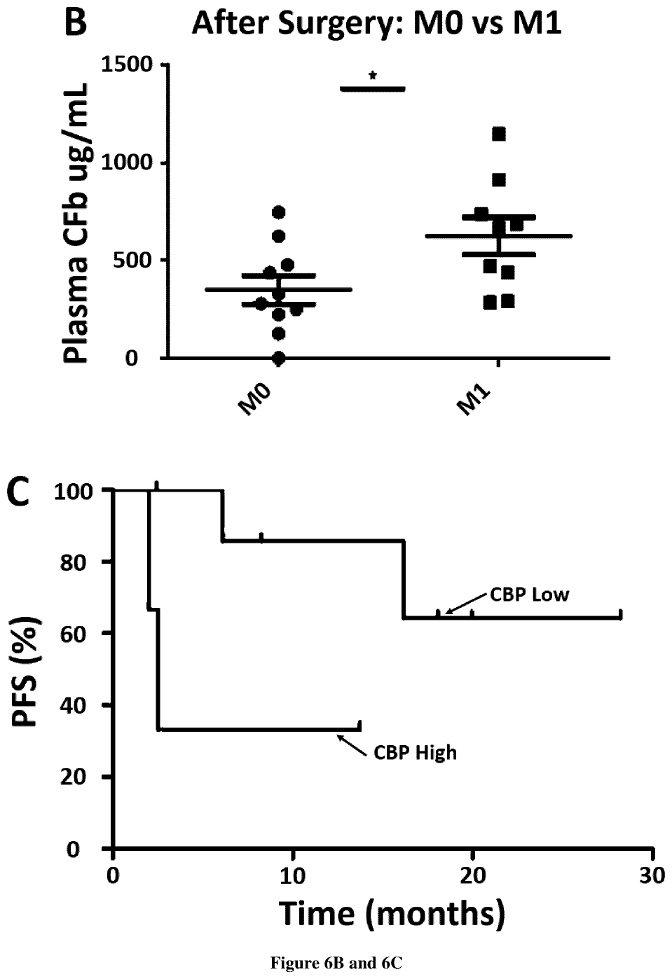

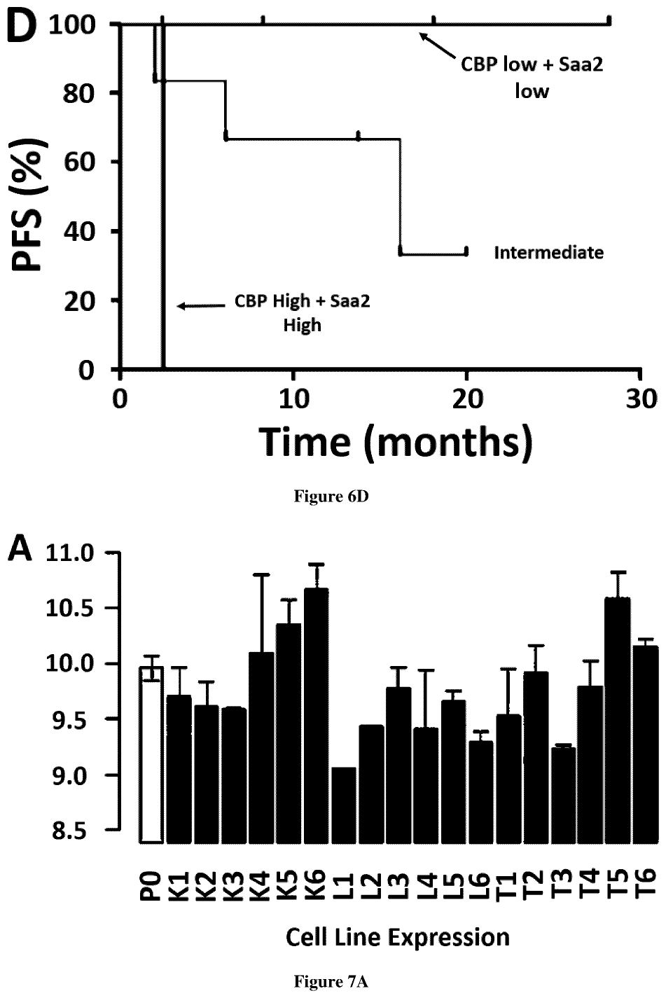

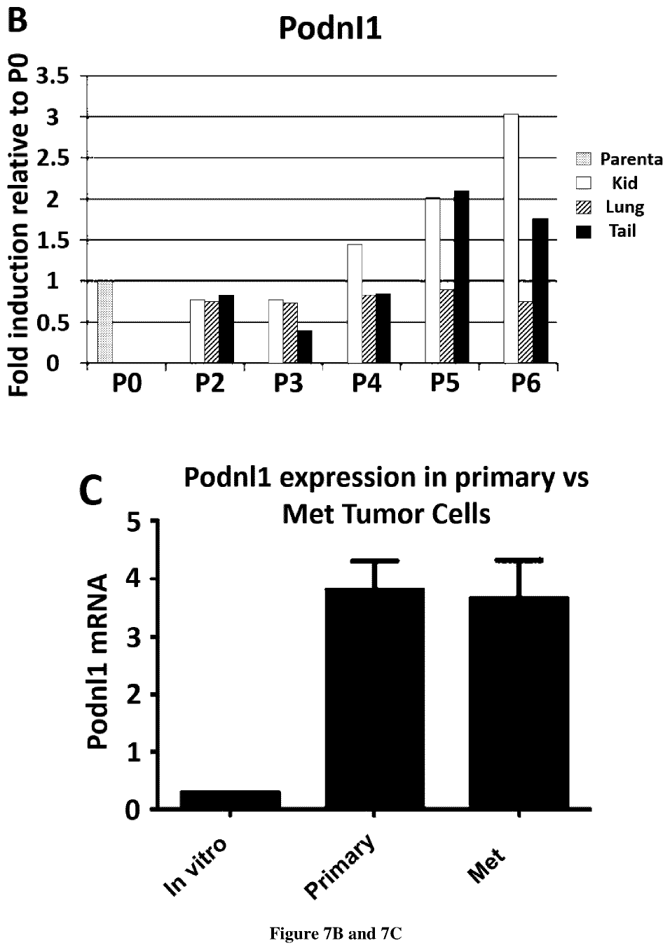

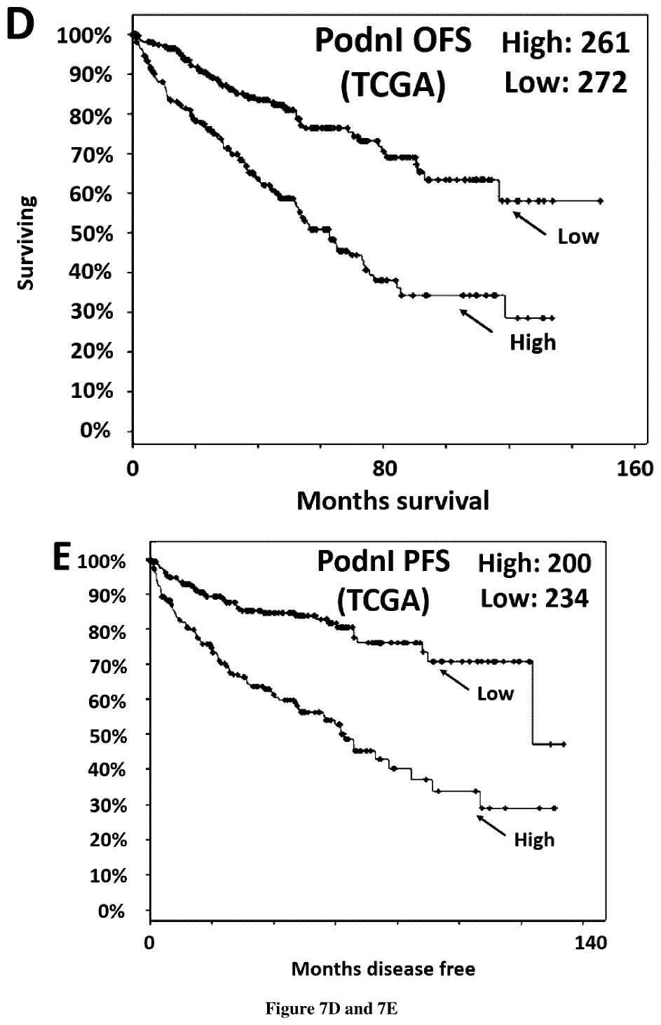

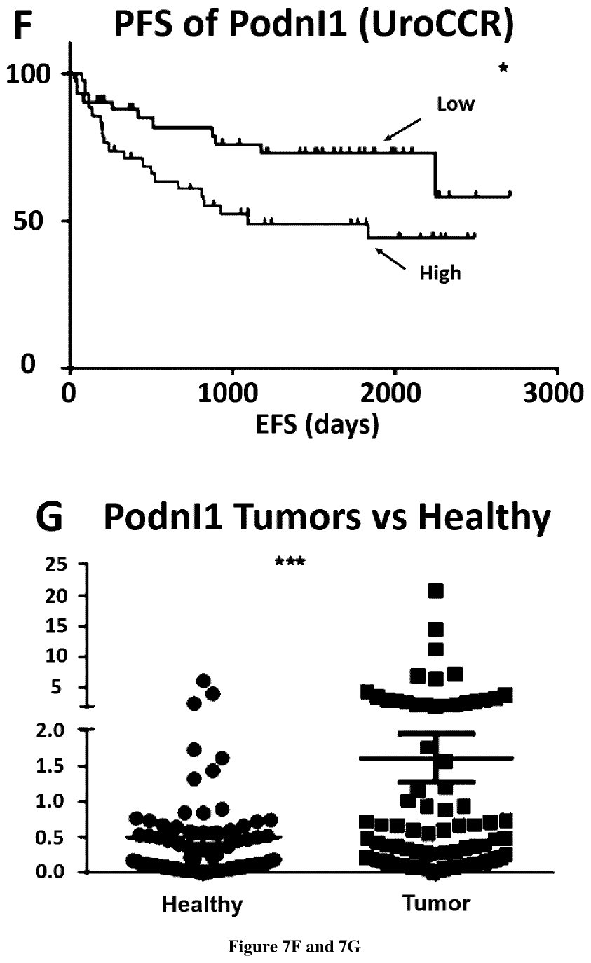

FIGS. 1A to 1I show the interleukin-34 (IL34) expression in mouse and human samples. FIGS. 2A to 2F show the IL34 Crispr-Cas9 deletion and response to Sutent treatment. FIG. 3A to 3G show the Serum Amyloid Protein A2 (SAA2) expression in mouse and human RCC samples. FIG. 4A to 4F show the Serum Amyloid Protein A2 (SAA2) in patient plasma/serum samples. FIG. 5A to 5G show the Complement Factor B (CFb) expression in mouse and human RCC samples. FIG. 6A to 6D show the Complement Factor B (CFb) in patient plasma/serum samples. FIG. 7A to 7G show the Podocan-like1 (Podnl1) expression in mouse and human RCC samples.

Serum Amyloid A2 (SAA2)

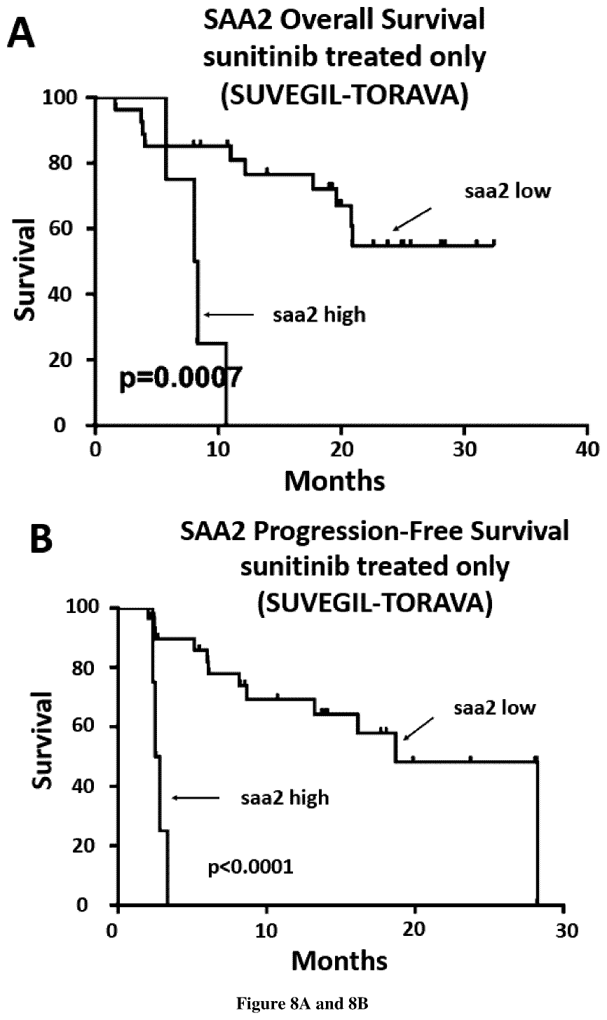

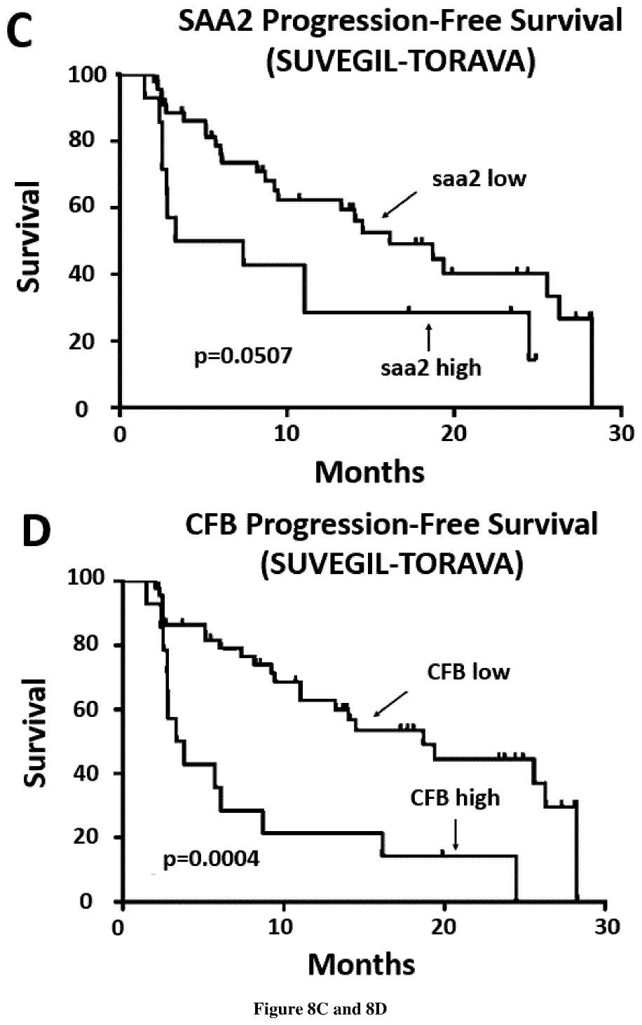

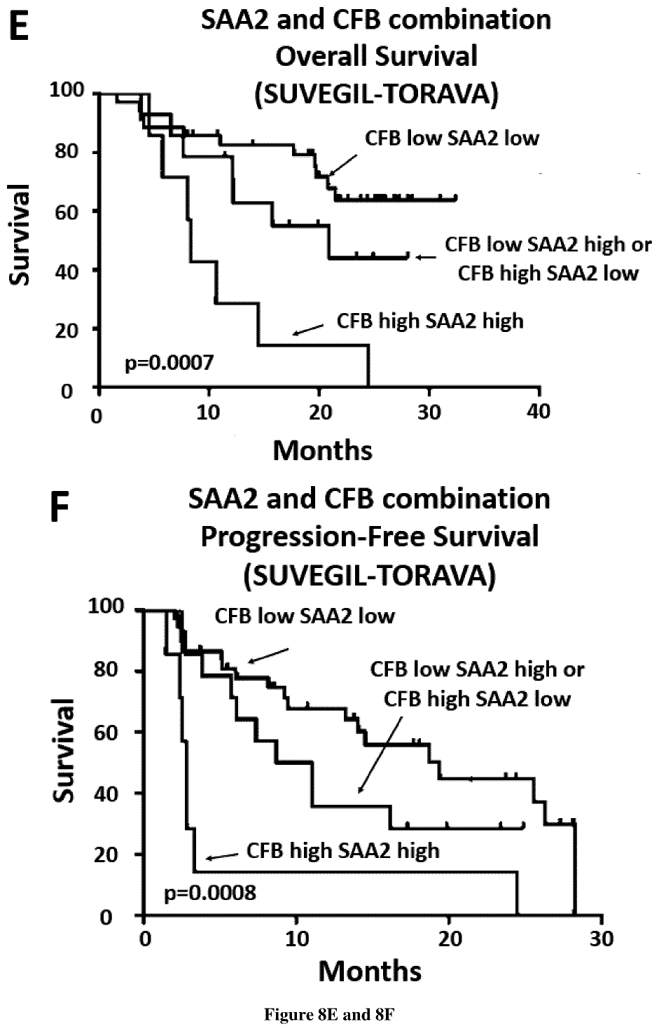

SAA2 is an acute phase protein related to SAA1, which was previously linked to metastasis. Its expression was strongly upregulated with passage in the Lung cell lines (data not shown). In silico analysis of the TCGA KIRC database SAA2 was a very strong predictor of OS (FIG. 3B) and DFS (FIG. 3C). Furthermore, the analysis was also done for the M0 and M1 subgroups (FIG. 3E). Analysis of the UroCCR patient cohort confirmed the effect on OS and DFS (FIG. 3G). Tumors from patients with the highest Fuhrman Grade, had a significantly increased SAA2 expression compared to all other grades (data not shown). We used grade-matched plasma samples from patients with and without metastases, collected before primary tumor surgery (data not shown). Patients with metastases had higher plasma levels of SAA2. When patients were divided into two groups of equivalent size, the group with higher SAA2 levels had a significantly shorter DFS (Supplementary FIG. 8l). A second set of plasma samples, collected in the weeks following surgery for removal of the primary tumor, was tested for SAA2 (Supplementary FIG. 8m). In this case, patients with higher expression had shorter OS. Hence, circulating SAA2 levels appear as an indicator of metastatic progression that deserves to be evaluated at diagnosis. We next used plasma samples from metastatic patients before receiving a first cycle of sunitinib or bevacizumab (SUVEGIL and TORAVA clinical trials). Patients treated with sunitinib only and stratified according to low and high SAA2 levels, had a spectacular better OS and progression-free survival (PFS) when belonging to the SAA2 low group (cut-off of 269 μg/ml) (FIG. 8A and 8B). When patients treated with sunitinib and bevacizumab were analyzed together, the PFS was of limited significance (borderline p-value of 0.0507) (FIG. 8C). The median of PFS for SAA2high patients was of 5.35 month versus 16.17 month for the SAA2low group. Thus, determining SAA2 plasma levels could be a useful measure for deciding a treatment strategy in RCC.

Complement Factor-B (CFB)

CFB was most strongly upregulated in the “Lung” and to a lesser extent in the “Tail” group, both considered to recapitulate features of metastasis (data not shown). TCGA analysis in ccRCC showed that CFB expression is correlated in primary tumors with shortened DFS and OS (FIG. 5D). We also performed the analysis in the M0 and M1 subgroups (data not shown). Using samples and data from the UroCCR cohort, we demonstrated that CFB was overexpressed in the tumor tissue versus the adjacent kidney at the mRNA level (FIG. 5E), and that increased expression correlated with reduced DFS and OS, consistent with the results obtained with the TCGA cohort (FIGS. 5F and 5G). As for SAA2, CFB can be measured in the blood. For this purpose, we used UroCCR plasma samples collected from patients either before surgery (primary tumor intact) or in the following weeks after surgery (no primary tumor present but metastases in situ possible). Before surgery, a trend was observed without reaching significance whereas after surgery patients with metastases had higher plasma CFB levels compared to patients without metastases (data not shown). This suggests that circulating CFB measurement may be useful as a blood-born marker of metastasis in the follow-up after surgical tumor removal. As for SAA2, CFB plasma levels were tested in patients with metastases before the first cycle treatment with sunitinib or bevacizumab (SUVEGIL and TORAVA clinical trials). Patients whose levels were high (cut-off 310 μg/ml) had faster disease progression compared to patients whose levels were low (high CFB, 3.58 month; low CFB, 18.7 month, p=0.0004) (FIG. 8D). We then grouped the significance of testing SAA2 and CFB plasmatic levels (FIGS. 8E and 8F). Three different groups with different survival can be identified: group 1 (CFB low+SAA2 low, PFS: 19.37 months, OS: NR), group 2 (CFB high SAA2 low or CFB low SAA2 high, PFS: 9.87 months, OS: 20.9 months), group 3 (CFB high Saa2 high, PFS: 2.8 month, OS:8.33 months). Group 1 had the best survival rate while group 3 had the worst. Group 2 had intermediate survival outcome. Thus, the combined analysis of these two markers is a powerful predictor of patient outcome following anti-angiogenic treatment with sunitinib or bevacizumab.

Podocan Like Protein-1 (PODNL1)

PODNL1 is a member of the small leucine-rich proteoglycan (SLRP) family of 17 genes. It is secreted extracellularly and its function is currently unknown. High expression has previously been linked with poor outcome in ovarian cancer and glioblastoma. PODNL1 expression was upregulated in our mouse cell lines in the “Kidney” subgroup (data not shown), although the increase was relatively modest. However, this gene showed a very strong link with reduced DFS and OS in the TCGA KIRC database (FIGS. 7D and 7E). We have also performed this analysis in M0 and M1 patients (data not shown). When using UroCCR samples, PODNL1 was overexpressed at the mRNA level in the tumor versus healthy tissue (FIG. 7G). In the UroCCR biobank, DFS also showed different trends depending on PODNL1 expression albeit statistically not significant, the latter was also the case for OS (FIG. 7F). This largely unknown and interesting gene may play a key role in RCC and further studies are required to investigate this possibility.

### REFERENCES

Throughout this application, various references describe the state of the art to which this invention pertains. The disclosures of these references are hereby incorporated by reference into the present disclosure.

1. A method for predicting the survival time of a patient suffering from a renal cell carcinoma (RCC) comprising i) determining the expression level of at least one biomarker selected from the group consisting of IL-34, SAA2, PONL1 and CFB in a sample obtained from the patient, ii) comparing the expression level determined at step i) with a predetermined reference value and wherein a difference between the determined expression level and said predetermined reference value is indicative whether the patient will have a long or short survival time.

2. The method of claim 1 wherein the sample is a blood sample (e.g. serum sample) or a tumor tissue sample.

3. The method of claim 1 wherein the expression levels of 2 biomarkers are determined in the sample.

4. The method of claim 1 wherein the expression levels of 3 biomarkers are determined in the sample.

5. The method of claim 1 wherein the expression levels of the 4 biomarkers (i.e. IL-34, SAA2, PONL1 and CFB) are determined in the sample.

6. The method of claim 1 wherein a score which is a composite of the expression levels of the different biomarkers is determined and compared to the predetermined reference value wherein a difference between said score and said predetermined reference value is indicative whether the patient will have a long or short survival time.

7. Use of the method of claim 1 for selecting a therapeutic regimen or determining if a certain therapeutic regimen is more appropriate for a patient identified as having a poor prognosis.

8. A method of treating RCC in a patient comprising
identifying the patient as having a poor prognosis by testing the patient according to the method of claim 1,
administering an anti-cancer therapy to the patient when the patient is identified as having a poor prognosis,
retesting the patient after the step of administering, and
administering the anti-cancer therapy to the patient at a maintenance dose when the patient is identified as having a good prognosis.

9. Use of the method of claim 1 for determining whether the patient will be responsive or experience a positive treatment outcome to a treatment.

10. A method of treating renal cell carcinoma (RCC) in a patient in need thereof comprising administering to the subject a therapeutically effective amount of an inhibitor of IL-34, SAA2, PONL1 or CFB.

11. The method of claim 10 wherein the treatment comprises administering to the patient an anti-VEGF agent.

12. The method of claim 10 wherein the patient was previously predicted as having a poor prognosis by the method of claim 1.

13. The method of claim 10 wherein the inhibitor is an antibody having specificity for IL-34, SAA2, PONL1 or CFB.

14. The method of claim 13 wherein the antibody is a chimeric antibody, a humanized antibody of a human antibody.

15. The method of claim 10 wherein the inhibitor is an inhibitor of expression.

16. The method of claim 15, wherein the inhibitor of expression is a siRNA or an antisense oligonucleotide.
